(19) United States
(12) Patent Application Publication
(10) Pub. No.: US 2023/0090966 A1
(43) Pub. Date: Mar. 23, 2023

(54) ULTRASOUND-BASED IMAGING DUAL-ARRAY PROBE APPARTUS AND SYSTEM

(71) Applicant: Rivanna Medical, Inc., Charlottesville, VA (US)

(72) Inventors: Frank William Mauldin, Jr., Troy, VA (US); Adam Dixon, Charlottesville, VA (US); Zachary Leonard, Charlottesville, VA (US)

(73) Assignee: Rivanna Medical, Inc., Charlottesville, VA (US)

(21) Appl. No.: 17/950,399

(22) Filed: Sep. 22, 2022

Related U.S. Application Data
(60) Provisional application No. 63/246,859, filed on Sep. 22, 2021.

(51) Int. Cl. A61B 8/12; A61B 90/00

(52) U.S. Cl. CPC A61B 8/12; A61B 90/37

(57) ABSTRACT

An ultrasound-based scanning apparatus and system using a dual-array probe and slot for guidance and insertion of a needle to assist with needle injection procedures.

### CROSS-REFERENCE TO RELATED APPLICATIONS

The present application relies on the disclosures of and claims priority to and the benefit of the filing date of U.S. Application No. 63/246,859, filed Sep. 22, 2021. The disclosures of the above application are hereby incorporated by reference herein in their entireties.

### STATEMENT REGARDING FEDERALLY SPONSORED RESEARCH

This invention was made with government support under Grant No. R44CA246854 awarded by the National Institutes of Health (NIH) National Cancer Institute (NCI). The government has certain rights in the invention.

### BACKGROUND

### Field of the Invention

The present invention is related to an ultrasound-based scanning device and more specifically to an apparatus and insertion method for using ultrasound-based scanning to assist needle injection procedures.

### Description of Related Art

Needle guidance procedures in medicine are numerous and comprise many different procedures including, for example, lumbar punctures, bone marrow biopsies, and chronic pain therapy injections. The techniques available for injection guidance range from a palpation-based approach, where no image guidance is utilized, to guidance under computed tomography or fluoroscopy. The palpation approach is low-cost and accessible at the bedside, yet suffers from low procedure success rates and higher rates of complications. Conventional ultrasound can improve success rates, and is utilized in some instances, but suffers from limitations including an extended learning curve and high level of operator dependence. X-ray-based approaches, such as computed tomography or fluoroscopy, exhibit high success rates but expose the patient to ionizing radiation, increase procedure cost, and are generally inaccessible at the bedside.

### SUMMARY OF THE INVENTION

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

The present invention overcomes limitations of existing needle guidance systems with, in aspects, large field of view images of anatomical targets, real-time needle visualization, and hands-free needle advancement. The invention, in aspects, utilizes a unique dual-array device for both two-dimensional and three-dimensional scanning, with a U-slot between the arrays that may act as or comprise a needle guide. Herein, the apparatus enables needle advancement in-plane with a real-time and/or simultaneous ultrasound image acquisition.

More specifically, to overcome the limitations of current state of the art approaches to medical needle guidance procedures, the present invention describes a unique ultrasound-based three-dimensional dual-array probe which can be mounted to a mechanical holding arm. The invention combines benefits of medical ultrasound, with no ionizing radiation and bedside portability, with advantages typical of X-ray-based modalities.

The apparatus uses the dual-array probe in combination with a linear actuator to generate a three-dimensional view of the insertion area or cavity of the patient. The apparatus additionally locks the dual-array probe in a home position during needle insertion. While locked in the home position, the dual-array probe generates a two-dimensional view of the insertion area or cavity of the patient. The computer processor overlays the two-dimensional and three-dimensional image for assisting needle insertion, along with the opening defined by the U-slot.

The novel dual-array three-dimensional probe allows for large field of field image acquisitions typical of fluoroscopy. A ‘U-slot’ between the two arrays enables in-plane needle insertion during contemporaneous image acquisition. This probe can be mounted to a mechanical holding arm such that a clinician may use both hands to advance the needle. This hands-free approach is an advancement compared to conventional ultrasound where the clinician requires one hand to hold the ultrasound imaging transducer and the second hand to advance the needle. In this way, the workflow and images provided to the clinician are substantially equivalent to fluoroscopy, but with the combined advances of utilizing an ultrasound-based imaging modality. Various preferred embodiments of the invention are described herein.

### BRIEF DESCRIPTION OF THE DRAWINGS

The accompanying drawings illustrate certain aspects of some of the embodiments of the present invention and should not be used to limit or define the invention. Together with the written description, the drawings serve to explain certain principles of the invention. For a fuller understanding of the nature and advantages of the present technology, reference is made to the following detailed description of preferred embodiments and in connection with the accompanying drawings, in which:

FIGS. 1A-1C are a schematic illustration of an exemplary imaging device.

FIGS. 2A-2D illustrates a cross-sectional illustration of the field of view of dual arrays.

FIG. 3 illustrates the imaging device mounted to a mechanical arm on a cart.

FIG. 4 illustrates an exploded view of an exemplary imaging device.

FIG. 5 is a flow diagram of one embodiment of a process for needle insertion.

### DETAILED DESCRIPTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Ultrasound imaging transducer assemblies are used in a variety of medical or clinical applications to enable medical imaging functions. In this non-limiting example, an ultrasound imaging transducer is disposed within a transducer assembly to deliver a pulse, tone, sequence, or programmed energy signal into a target location to be imaged. A specific example is one or more ultrasound transducer elements that deliver an ultrasound signal into a patient's body and detect a return signal so as to form a computer-generated image of the target region. Different ultrasound imaging modes can be utilized, depending on a given application and design as known to those skilled in the art.

The present disclosure can be used in medical ultrasound applications but is not limited to this application. Those skilled in the art will appreciate that a variety of types of transducers, signal transmitters and/or receivers, and other arrays can also benefit from the present invention, which are comprehended hereby.

The present apparatus provides for inserting a medical instrument into a patient, more specifically into a desired or defined insertion cavity of the patient. In one embodiment, the medical instruction is a needle, the apparatus providing needle insertion guidance. Those skilled in the art will appreciate that the present invention may be used to guide a variety of medical instruments including, but not limited to, a catheter, trocar, ablation instrument, or therapy applicator. Therefore, the insertion of a needle is one exemplary embodiment and not expressly limiting where noted herein.

The present invention can be utilized, in a preferred embodiment, with systems and methods previously disclosed by Mauldin et al. (PCT/US2019/012622), the entirety of which is incorporated by reference herein, including disclosures for automated three-dimensional detection, guidance, and visualization of ultrasound-based therapy guidance procedures.

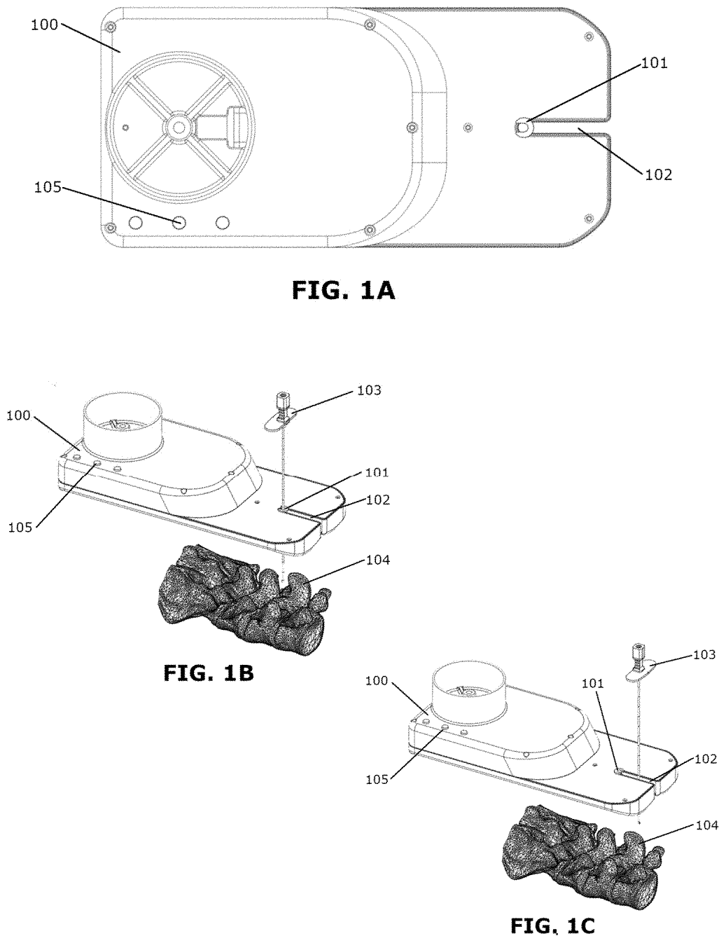

In one embodiment, an ultrasound imaging device 100 is depicted in FIG. 1A. The device shown has a longitudinal slot 102 to permit needle placement. In one embodiment, the slot 102 is a U-shaped slot. The longitudinal slot 102, in one embodiment, includes a needle guide 101. As illustrated in FIG. 1A, the needle guide 101 is disposed an interior position within the longitudinal slot 102. Herein, the needle guide is positioned for assisting inserting the needle at the desired anatomical location of the patient.

In further embodiments, the device 100 includes user interface elements, including for example buttons 105. As described in greater detail below, the buttons 105 or other user interface elements, facilitate operations of the device 100 both prior to and during needle insertion. Non-limiting examples of the user interface used to control and interact with the imaging device include buttons 105, an LCD screen, a touch screen or similar interface(s) as recognized by one skilled in the art.

As noted herein, the device 100 provides for inserting the medical instrument into the patient. FIG. 1B illustrates the imaging device 100 in a schematic illustration of needle insertion into a patient's spinal anatomy. The needle guide 101 constrains the location of the needle 103 at the base of the longitudinal slot 102 to allow accurate needle placement in the desired insertion cavity, here being an anatomic location of the spinal anatomy 104. Further visible, apparatus 100 includes the user interface 105.

FIG. 1C illustrates the imaging device 100 in a schematic illustration of needle removal from a patient's spinal anatomy in FIG. 1C. The needle guide 101 may allow removal of the needle 103 from the spinal anatomy 104 and imaging device 100 through the length of the longitudinal slot 102. The needle guide 101 can be configured to either constrain or allow removal of the needle 103 through mechanisms including but not limited to rotation, opening, or removal of the needle guide 101. In embodiments, the needle guide can incorporate a quick-release clasp mechanism that can secure the needle in place when the clasp is closed and allows for removal of the needle when it is opened. In such embodiments, the needle guide may be fabricated as a single injection molded plastic component that incorporates the clasp mechanism and quick-release locking features, including serrated edges and pawls, to maintain the clasp in its closed position.

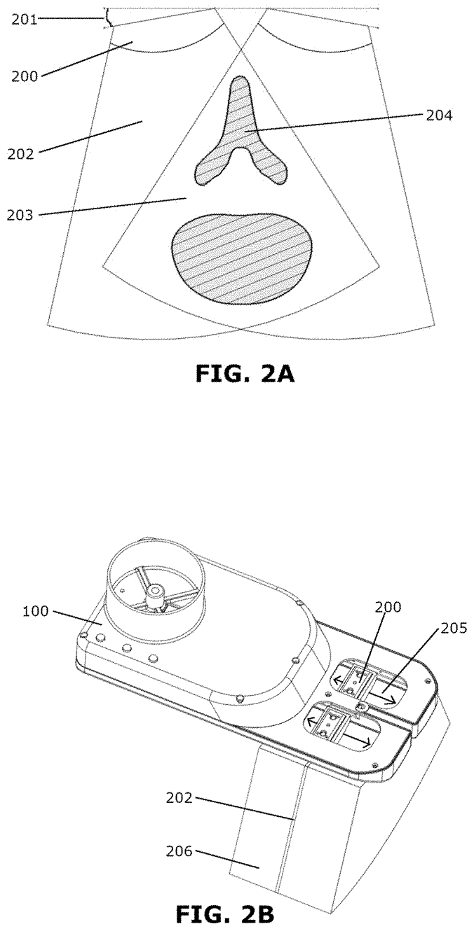

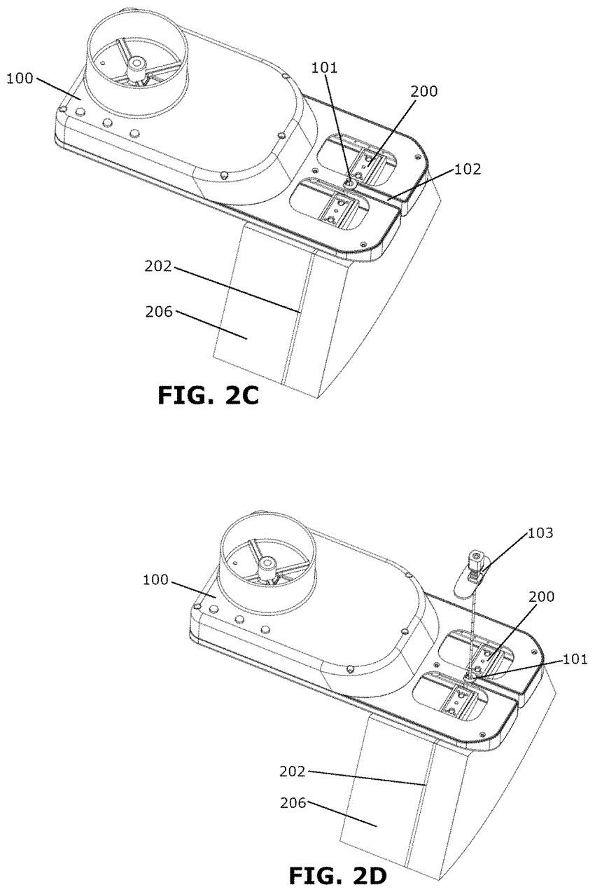

Enclosed within the imaging device 100 is a dual-array probe include two ultrasound transducer arrays. Using known operational techniques, transducer arrays operate to generate ultrasound readings. FIG. 2A illustrates two ultrasound transducer arrays 200 positioned adjacent to each other such that a 2D ultrasound field of view 202 of each array overlaps in the central region 203. When applied relative to a patient, such as illustrated in FIG. 1B, the field of view 202 includes imaging of the target anatomy 204 from multiple angles of incidence.

In one embodiment, the, the arrays 200 may be optionally tilted by an angle 201 to modify the spatial position of the ultrasound field of view 202 and adjust the size and position of the overlapping central region 203. For example, the angle 201 can be an angle ranging from 1% to upwards of 65% or so, wherein the greater the angle 201 the smaller the field of view 202 or closer the insertion cavity to the device (100 of FIG. 1).

As for the facilitating inserting of the medical instrument, the apparatus includes processor operations for combining the 2D images acquired by each array 200 to reconstruct a composite 2D ultrasound image that fully encompasses the anatomical region spanned by the two individual fields of view 202.

FIG. 2B illustrates the apparatus 100 of FIG. 1, with a cross-section cut-out of the front end portion. In the embodiment shown in FIG. 2B, the ultrasound imaging device 100 contains two arrays 200 that can be linearly translated along an axis 205. Within the apparatus 100 is a linear actuator for moving the ultrasound transducers 200 along the designated axis 205.

While moving the transducers 200 along the axis 205, this enables acquisition of individual 2D imaging slices 202. For reference, the slice 202 is visible in FIG. 2A from a front view, as well as an angled view in FIG. 2B. The generation of multiple 2D image slices, as varying positions from the movement of the transducers 205 by the linear actuator, are combined to form a 3D volume 206 composed of multiple individual 2D imaging slices 202. As visible in FIG. 2B, the slice 202 is a single 2D image and the 3D image or volume 206 is a combination of multiple slices generated by movement of the transducer arrays 200. As described in further detail herein, the generation of the 2D and 3D images are performed by processor operations processing image data acquired from the arrays 200.

The apparatus 100 can operate at two different stages or phases. In the first stage, the apparatus 100 generates the 3D imagery with movement of the transducer arrays 200. In the second stage, the apparatus 100 facilitates needle insertion into the patient.

When a position for needle insertion is known, the transducer arrays 200 are looked in a set position, this is referred to as a “home” position. While in the home position, the arrays 200 generate 2D imagery, but this 2D imagery is a single slice, e.g. 202. As shown in FIG. 2C, the arrays 200 can be held stationary at a home position that aligns the ultrasound array fields of view 202 with the axis of needle insertion that is imposed by the needle guide 101.

Positioning the arrays 200 at the home position allows the imaging device 100 to capture real-time images of the 2D ultrasound field of view 202 to allow accurate placement of a needle 103 in the target anatomy as illustrated in FIG. 2D. As the needle 103 is inserted into the patient and towards the insertion cavity, an output display provides an imagery of the 2D imagery actively being generated by the transducer arrays 200 in the home position in an overlay with the 3D imagery acquired prior to locking the transducer array 200 in the home position.

In one or more embodiments, an indication of a three-dimensional anatomical structure location is displayed as two-dimensional anatomical structure images with a third dimension encoded to represent an anatomical structure location along the third dimension. In one or more embodiments, the third dimension is graphically encoded to represent the anatomical structure location along the third dimension. In one or more embodiments, the third dimension is color encoded to represent the anatomical structure location along the third dimension. In one or more embodiments, two-dimensional ultrasound image data of the anatomy is acquired at a plurality of locations by the ultrasound transducer arrays; and combining the two-dimensional ultrasound image data and the ultrasound transducer arrays locations to form the three-dimensional image data. In one or more embodiments, the two-dimensional image data includes pixels, wherein a three-dimensional position of each pixel based on the ultrasound transducer arrays locations is determined.

In one or more embodiments, the appropriate position and the appropriate orientation of the needle are determined based at least in part on predetermined relative position(s) of the needle with respect to the ultrasound imaging device with adjacent ultrasound transducer arrays. In one or more embodiments, an object tracker can be configured to detect the current position and the current orientation of the needle, and the appropriate position and the appropriate orientation of the needle are determined based at least in part on the current position and the current orientation of the needle.

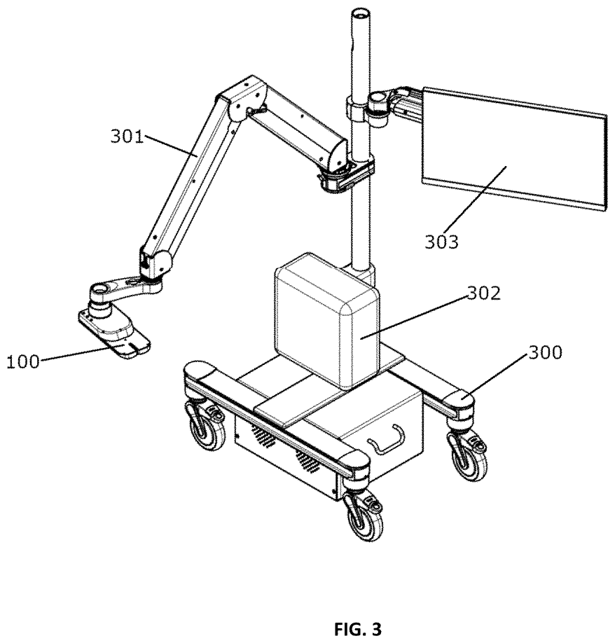

In an embodiment depicted in FIG. 3, the ultrasound imaging device 100 can be mounted to an adjustable mechanical arm 301 on a mobile cart 300 to allow the imaging device to be moved to the bedside and positioned at the required orientation for acquiring images of the patient's anatomy, including the desired anatomical location for needle insertion. The adjustable mechanical arm 301 may be self-balancing, powered, or robotically controlled among other embodiments known to those skilled in the art. The adjustable mechanical arm 301 may comprise a locking feature to rigidly affix the imaging device 100 in place for image acquisition and needle advancement. The cart 300 shown includes an enclosure 302 which may contain a computer, battery, and other associated electronics familiar to those skilled in the art which are needed to power and communicate with the imaging device 100. The cart 300 can be outfitted with additional input/output devices such as a keyboard, mouse, or monitor 303, which may also be a touchscreen display.

Both adjustable mechanical arm 301 and monitor 303 components may be positionally adjustable about the cart in order to orient the imaging device 100 and monitor in various relative positions for the needle guidance procedure. In a preferred embodiment, the enclosure 302 may additionally contain ultrasound front-end electronics.

A computer with computer processor within the enclosure 302 may be used to perform ultrasound signal and image processing steps required to form an ultrasound image reconstruction that can be displayed on the monitor 303. Such processing steps are known to those skilled in the art of medical ultrasound and may include beamforming, bandpass filtering, scan conversion, and image rendering among other processes. Two- and three-dimensional images may be rendered using various techniques, including simultaneous display, as described in Mauldin et al. (PCT/US2019/012622).

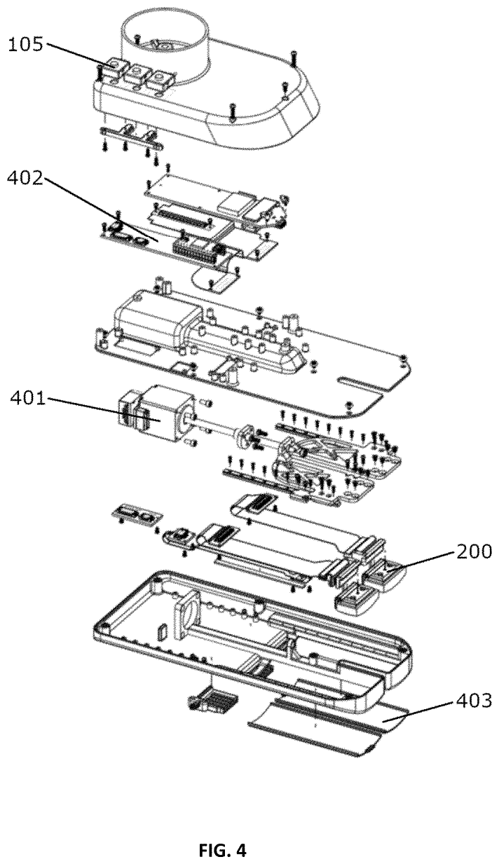

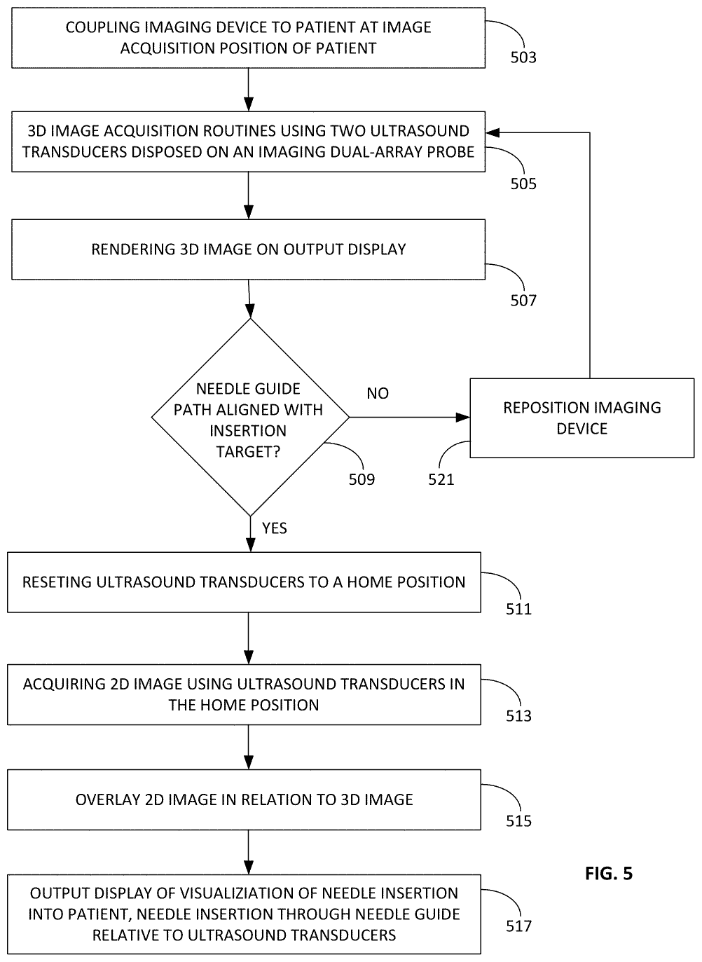

An exploded, component-level view of an exemplary imaging device is depicted in FIG. 4. The depicted imaging device contains ultrasound arrays 200, a linear actuator 401, control and acquisition electronics 402, user interface buttons 105, and associated electromechanical components, which are packaged in mechanical housings. Control and acquisition electronics 402 may incorporate a position encoder with a computer processor to instruct linear position changes to the linear actuator 401, image acquisition from the ultrasound arrays 200, and signal or image processing steps applied to the acquired ultrasound image signals. The user interface buttons 105 may initiate functions including full three-dimensional acquisitions or a “home” function that places the arrays 200 at a defined home position corresponding to the needle guide path 101. Other functions may include instructions for the system computer to save an image acquisition to the patient record, pause imaging, or change imaging parameters. The imaging device includes acoustic lenses 403 and acoustic couplants, which may be discrete parts or integral to other mechanical housing(s), for optimized transmission of acoustic energy from the arrays 200. Additional ultrasound arrays, matrix transducer arrays, or C-MUT arrays may be used in place of the two ultrasound arrays 200 depicted in FIG. 4 in order to improve field of view or image acquisition speeds. The imaging device may include LED indicators 404 that indicate the current position of the arrays 200 along the linear path driven by the linear actuator 401. Other indicators may include a transparent plastic slot allowing the using to visibly observe the array position 200, an LCD display, or other indicators as known to those skilled in the art. The indicators may also have functions including an indication of power on/off, image capture, or arrival of the arrays 200 at the home position.

The above-described ultrasound imaging and therapy guidance system is operable in an injection methodology. The ultrasound imaging and therapy guidance system provides for improved accuracy and efficacy of injection operations. FIG. 5 illustrates a flowchart of one embodiment of a methodology for ultrasound-based injection operations.

Step 503 is coupling the imaging device to a patient at an image acquisition position. The coupling provides for disposition of the imaging device, for example imaging device 100 noted above, in contacting engagement with the patient including using ultrasound coupling gel or other lubricant compatible with medical ultrasound and known to those skilled in the art.

Step 505 includes execution of 3D image acquisition operations. In one embodiment, the 3D image acquisition routines are executed by the dual ultrasound transducers noted above. In one embodiment, the 3D image acquisition routine can be initiated by the user. Initiation may be achieved through the monitor 303 of FIG. 3, a user input button or command can be associated with or engageable on the monitor 303, for example a touch screen interface on the screen 303. Initiation may be achieved by other user input operations, including but not limited to user interface buttons 105 of FIG. 1, or by other means as recognized by one skilled in the art.

Step 507 is rendering 3D images. As noted above, the 3D image acquisition is achieved by synchronous translation of the arrays 200 of FIGS. 2A-2D, which are driven by the linear actuator 401, and ultrasound image acquisitions instructed by the control and acquisition electronics 402 of FIG. 4. As noted relative to FIG. 3, the ultrasound image acquisitions are transmitted to the computer system within the enclosure 302 and the reconstructed image is displayed on the monitor 303.

Step 509 is an assessment step, determining if the alignment of the needle guide path 101 of FIG. 1, conforms with the target anatomy, also referred herein as the insertion cavity of the patient based as a desired anatomical location. This assessment may be produced automatically by a processing algorithm run on the computer system, such as described by Mauldin et al. (PCT/US2019/012622), or it may be achieved by the user through visual assessment of the rendered imaging results.

If the determination in step 509 is in the negative, i.e. where there is not alignment, the method reverts to step 521. Step 521 is re-positioning the imaging device, e.g. device 100 of FIG. 1, and may include re-coupling of the imaging device to the patient, as needed. Repositioning the device may be done manually by an operator or in another embodiment can be performed using motorized controls associated with the cart visible in FIG. 3. Upon repositioning, the method reverts to step 505, repeating steps 505 and 507 until the inquiry of step 509 is in the affirmative.

Upon determining the needle guide path aligns with the insertion target of the insertion cavity of the patient, the method reverts to step 511. In step 511, the ultrasound transducers are reset to a home position. These transducers may be dual ultrasound transducers noted above, e.g. transducers 200 of FIGS. 2A-2D. As described herein, at the home position the arrays 200 are oriented such that the two-dimensional image acquisition is co-aligned with the needle guide path 101. The reset to a home position includes electrical and computer processing operations to designate the base position for the transducers and the imaging device for needle insertion operations.

Step 513 is two-dimensional image acquisition processing operations performed by the imaging system. These image acquisition processing operations utilize the processing device(s) and executable software code as noted above. The execution of the processing operations and generation of the 2D image provides a technical solution for image alignment and needle insertion using operations building upon the prior steps of the present methodology.

Step 515 provides for overlay of the 2D image and the 3D image. In one embodiment using the embodiment of FIG. 3, the images are processed by computing devices within element 302 and visible on the output display 303. Three-dimensional image renderings and two-dimensional real-time image acquisitions are displayed on the monitor 303. The integration of the display 303 with image processing operations 302 facilitates further needle insertion.

In step 517, the user advances the needle through the needle guide, such as needle guide 101 of FIG. 1A-FIG. 1C. As part of the image processing routines using computerized processing operations performed by the computer processing device, the needle is visualized within the ultrasound image rendering displayed on the monitor 303 as it advances to the needle target. Herein, the methodology of FIG. 5 provides a technical solution of generating and overlaying 2D and 3D imagery for facilitating needle insertion using the imaging device coupled to the patient.

In one or more embodiments, images generated by an electronic display include the current position and the current orientation of the needle. In one or more embodiments, the images generated by the display further include dimensional and orientation information of the desired anatomical structure or location (or other anatomy) calculated from the anatomical surface locations and image data provided by the ultrasound transducer arrays.

In one or more embodiments, the therapy applicator comprises a needle and/or an ablation instrument, in one or more embodiments, a desired anatomical location can be an anatomical structure or surface location, an anatomical structure, an anatomical surface, a target therapy site, an epidural space, an intrathecal space, or a medial branch nerve, by way of non-limiting example only. In one or more embodiments, the device is configured to be positionally adjusted manually by a user, in one or more embodiments, the device is configured to be positionally adjusted automatically with a mechanical motorized mechanism.

In one or more embodiments, the object tracker includes inductive proximity sensors. In one or more embodiments, the object tracker includes an ultrasound image processing circuit. In one or more embodiments, the ultrasound image processing circuit is configured to determine a relative change in the current position of the device by comparing sequentially-acquired ultrasound images of the image data, such as three-dimensional image data.

In one or more embodiments, the object tracker includes optical sensors. In, one or more embodiments, the optical sensors include fixed optical transmitters and swept lasers detected by the optical sensors, the optical sensors disposed on the device. In one or more embodiments, the object tracker includes integrated positioning sensors. In one or more embodiments, the integrated positioning, sensors include an electromechanical potentiometer, a linear variable differential transformer, an inductive proximity sensors, rotary encoder, an incremental encoder, an accelerometer, and/or a gyroscope. In one or more embodiments, anatomical structure or anatomical surface structure locations include three-dimensional spine bone locations.

Aspects of the invention are directed to an ultrasound-based imaging dual-array probe combined with three-dimensional (3D) position tracking that enables highly accurate 3D surface rendering for the purpose of anatomical assessment and/or procedure guidance (e.g., to guide a therapy applicator such as a needle and/or an ablation device during energy-based ablation). In aspects, a 3D anatomical structure image can be generated by tracking (e.g., with a position tracking system) the spatial location, and optionally the orientation, of the ultrasound-based imaging dual-array probe as it is positionally adjusted proximal to a target area on a human subject, such as an anatomical structure or desired anatomical structure (e.g., to acquire image data of anatomy proximal to a target area for the therapy applicator). The 3D anatomy image, such as 3D bone image, can be automatically annotated such as by providing indications of joint or bony feature locations, bone fracture locations, indications of optimal needle insertion areas or angles, indication of possible needle or therapy sites, and/or indications and degree of scoliosis and/or other bony anatomy abnormalities.

In aspects, real-time feedback can be provided to the user during scanning by the ultrasound-based imaging dual-array probe (e.g., while the 3D anatomical structure image is acquired) so that 3D anatomy proximal to, for example, a target area, is scanned in all locations and/or orientations required to provide a 3D display of the reconstructed anatomical structure (e.g., bone, spine, or cavity with annotations and/or model fitting information.

In aspects, the position tracking system tracks the therapy applicator (e.g., needle) in addition to the ultrasound-based imaging dual-array probe. After the 3D anatomical structure information is constructed the system or device can provide real-nine guidance of the therapy applicator to a desired location/structure. For example, the therapy applicator can be a needle, a catheter, or a radiofrequency ablation probe. A desired therapy site, anatomical structure, or desired anatomical structure could be a bone, a spine, the epidural space, a facet joint, or a, sacroiliac joint, by way of non-limiting example only. In some embodiments, the real-nine guidance can include guiding the therapy applicator while the therapy is being applied, such as during an energy-based ablation. The desired location/structure can be a location specified by the user, such as by indicating a location on the 3D structure or surface reconstruction where therapy should be applied. The system/device could then guide the therapy applicator to the location required in order for the desired therapy site to receive the therapy when the therapy applicator is activated. Alternatively, the location can be automatically provided by the system/device. The location can be an optimal location for the therapy applicator to accurately deliver the therapy to the desired therapy site, or could provide several choices for an optimal location (for example, at different intervertebral spaces).

The invention herein includes several Aspects, including but not limited to the following:

Aspect 1: An apparatus for inserting a medical instrument into a patient using ultrasound-based guidance, the apparatus comprising:

an ultrasound-based imaging dual-array probe comprising two ultrasound transducer array and

a longitudinal slot, the two ultrasound transducer arrays disposed on opposing sides of the longitudinal slot; and

a linear actuator for moving the ultrasound transducer arrays for generating imaging of an insertion cavity of the patient;

wherein the longitudinal slot and the imaging generated by the ultrasound transducer arrays provide in-plane guidance for insertion of the medical instrument into the insertion cavity of the patient at a desired anatomical location.

Aspect 2: The apparatus of Aspect 1, wherein the medical instrument is chosen from one of: a needle, a catheter, and an ablation instrument.

Aspect 3: The apparatus of Aspect 1, wherein the longitudinal slot acts as a guide for the insertion of the medical instrument.

Aspect 4: The apparatus of Aspect 1, wherein the longitudinal slot is a U-shaped slot.

Aspect 5: The apparatus of Aspect 1 further comprising:

a needle guide disposed within the longitudinal slot, the needle guide providing for in-plane insertion of the medical instrument into the patient at the insertion cavity by constraining a location of the medical instalment relative to the longitudinal slot.

Aspect 6: The apparatus of Aspect 1, wherein the movement of the ultrasound transducer arrays generates the imaging being a three-dimensional image of the insertion cavity.

Aspect 7: The apparatus of Aspect 6 further comprising:

a locking mechanism for securing the ultrasound transducer arrays in a home position, wherein when the ultrasound transducer arrays are in the home position, the ultrasound transducer arrays generate the imaging being a two-dimensional image of the insertion cavity.

Aspect 8: The apparatus of Aspect 7, wherein the in plane guidance for insertion of the medical instruction includes an overlap of the three-dimensional image and the two-dimensional image of the insertion cavity.

Aspect 9: The apparatus of Aspect 1, wherein the ultrasound transducer arrays are disposed at an angle relative to the insertion cavity of the patient.

Aspect 1.0 The apparatus of Aspect 1, wherein a first image from a first ultrasound transducer array of the two ultrasound transducer arrays has different imaging properties chosen from at least one of ultrasound frequency, beam angulation, focal depth, pressure amplitude, scanline density, and elevational scan plane, from a second image from a second ultrasound transducer array of the two ultrasound transducer arrays.

Aspect 11: The apparatus Aspect 1, further comprising a computer processor, wherein the computer processor generates the imaging of the insertion cavity of the patient including generating a first image from a first ultrasound transducer array of the two ultrasound transducer arrays and a second image from a second ultrasound transducer array of the two ultrasound transducer arrays and combining the first image and the second image to construct a composite two-dimensional ultrasound image of the insertion cavity.

Aspect 12: The apparatus of Aspect 1, further comprising a computer processor, wherein the computer processor generates the imaging of the insertion of cavity of the patient including generating a plurality of first images and a plurality of second images from the two ultrasound transducer arrays as they are linearly translated along an axis by the linear actuator, the computer processor constructing a three-dimensional ultrasound image based on the plurality of first transducer array images and the second transducer array images.

Aspect 13: The apparatus of Aspect 1 further comprising:

a mounting assembly such that the apparatus is mounted to an adjustable mechanical holding arm allowing for hands-free adjustment of a position of the apparatus relative to the patient.

Aspect 14: The apparatus of Aspect 13, wherein the adjustable mechanical holding arm is attached to a cart.

Aspect 15: The apparatus of Aspect 13, wherein the adjustable mechanical holding arm is at least one of self-balancing and robotically-controlled.

Aspect 16: The apparatus of Aspect 13, further comprising a monitor, wherein both the adjustable mechanical arm and the monitor are positionally adjustable about the cart to orient the apparatus and monitor in various relative positions for a medical instrument guidance procedure.

Aspect 17: An ultrasound imaging and medical instrument guidance system comprising:

an ultrasound-based imaging dual-array probe comprising two ultrasound transducer arrays located adjacent to one another that generate two or more positionally-adjusted ultrasound beams to acquire image data of an anatomical structure in a patient, wherein a slot is provided between the two adjacent ultrasound transducer arrays for at least one of guiding the medical instrument, inserting the medical instrument into a cavity of a patient, removing the medical instrument from a cavity of the patient, and rotating the medical instrument;

a computer processor for combining at least two two-dimensional images from the adjacent ultrasound transducer arrays and displaying those two images as a single three-dimensional image of the anatomical structure using an electronic display;

a non-transitory computer memory operatively coupled to the computer processor, the non-transitory memory comprising computer-readable instructions that cause the processor to detect a position and an orientation of the anatomical structure based on the image data and a current position and a current orientation of the ultrasound-based imaging dual-array probe;

the display in electrical communication with the computer processor, the display generating images based on the image data, the images comprising an image of a location and orientation of the anatomical structure relative to a location and orientation of the medical instrument.

Embodiments of the invention also include a computer readable medium comprising one or more computer files comprising a set of computer-executable instructions for performing one or more of the calculations, steps; processes; and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. An apparatus for inserting a medical instrument into a patient using ultrasound-based guidance, the apparatus comprising:
an ultrasound-based imaging dual-array probe comprising two ultrasound transducer arrays; and
a longitudinal slot, the two ultrasound transducer arrays disposed on opposing sides of the longitudinal slot; and
a linear actuator for moving the ultrasound transducer arrays for generating imaging of an insertion cavity of the patient;
wherein the longitudinal slot and the imaging generated by the ultrasound transducer arrays provide in-plane guidance for insertion of the medical instrument into the insertion cavity of the patient at a desired anatomical location.

2. The apparatus of claim 1, wherein the medical instrument is chosen from one of: a needle, a catheter, and an ablation instrument.

3. The apparatus of claim 1, wherein the longitudinal slot acts as a guide for the insertion of the medical instrument.

4. The apparatus of claim 1, wherein the longitudinal slot is a U-shaped slot.

5. The apparatus of claim 1 further comprising:
a needle guide disposed within the longitudinal slot, the needle guide providing for in-plane insertion of the medical instrument into the patient at the insertion cavity by constraining a location of the medical instrument relative to the longitudinal slot.

6. The apparatus of claim 1, wherein the movement of the ultrasound transducer arrays generates the imaging being a three-dimensional image of the insertion cavity.

7. The apparatus of claim 6 further comprising:
a locking mechanism for securing the ultrasound transducer arrays in a home position, wherein when the ultrasound transducer arrays are in the home position, the ultrasound transducer arrays generate the imaging being a two-dimensional image of the insertion cavity.

8. The apparatus of claim 7, wherein the in-plane guidance for insertion of the medical instruction includes an overlap of the three-dimensional image and the two-dimensional image of the insertion cavity.

9. The apparatus of claim 1, wherein the ultrasound transducer arrays are disposed at an angle relative to the insertion cavity of the patient.

10. The apparatus of claim 1, wherein a first image from a first ultrasound transducer array of the two ultrasound transducer arrays has different imaging properties chosen from at least one of ultrasound frequency, beam angulation, focal depth, pressure amplitude, scanline density, and elevational scan plane, from a second image from a second ultrasound transducer array of the two ultrasound transducer arrays.

11. The apparatus claim 1, further comprising a computer processor, wherein the computer processor generates the imaging of the insertion cavity of the patient including generating a first image from a first ultrasound transducer array of the two ultrasound transducer arrays and a second image from a second ultrasound transducer array of the two ultrasound transducer arrays and combining the first image and the second image to construct a composite two-dimensional ultrasound image of the insertion cavity.

12. The apparatus of claim 1, further comprising a computer processor, wherein the computer processor generates the imaging of the insertion of cavity of the patient including generating a plurality of first images and a plurality of second images from the two ultrasound transducer arrays as they are linearly translated along an axis by the linear actuator, the computer processor constructing a three-dimensional ultrasound image based on the plurality of first transducer array images and the second transducer array images.

13. The apparatus of claim 1 further comprising:
a mounting assembly such that the apparatus is mounted to an adjustable mechanical holding arm allowing for hands-free adjustment of a position of the apparatus relative to the patient.

14. The apparatus of claim 13, wherein the adjustable mechanical holding arm is attached to a cart.

15. The apparatus of claim 13, wherein the adjustable mechanical holding arm is at least one of self-balancing and robotically-controlled.

16. The apparatus of claim 13, further comprising a monitor, wherein both the adjustable mechanical arm and the monitor are positionally adjustable about the cart to orient the apparatus and monitor in various relative positions for a medical instrument guidance procedure.

17. An ultrasound imaging and medical instrument guidance system comprising:
an ultrasound-based imaging dual-array probe comprising two ultrasound transducer arrays located adjacent to one another that generate two or more positionally-adjusted ultrasound beams to acquire image data of an anatomical structure in a patient, wherein a slot is provided between the two adjacent ultrasound transducer arrays for at least one of guiding the medical instrument, inserting the medical instrument into a cavity of a patient, removing the medical instrument from a cavity of the patient, and rotating the medical instrument;
a computer processor for combining at least two two-dimensional images from the adjacent ultrasound transducer arrays and displaying those two images as a single three-dimensional image of the anatomical structure using an electronic display;
a non-transitory computer memory operatively coupled to the computer processor, the non-transitory memory comprising computer-readable instructions that cause the processor to detect a position and an orientation of the anatomical structure based on the image data and a current position and a current orientation of the ultrasound-based imaging dual-array probe; and
the display in electrical communication with the computer processor, the display generating images based on the image data, the images comprising an image of a location and orientation of the anatomical structure relative to a location and orientation of the medical instrument.
